(19) United States
(12) Patent Application Publication
(10) Pub. No.: US 2006/0270914 A1
(43) Pub. Date: Nov. 30, 2006

(54) Apparatus for pediatric resuscitation

(76) Inventor: Marc-David Munk, Pittsburgh, PA (US)

Correspondence Address: Marc-David Munk;Apt 4, 707 College Ave, Pittsburgh, PA, 15232, US

(21) Appl. No.: 11/327,810

(22) Filed: Jan. 6, 2006

Related U.S. Application Data
(60) Provisional application No. 60/670,428, filed on Apr. 13, 2005.

(51) Int. Cl. A61B 5/00

(52) U.S. Cl. 600/300

(57) ABSTRACT

A medical apparatus is described that simplifies decision-making at the time of pediatric resuscitation by measuring the patient's weight while laying in the medical apparatus and using that information to reduce the need to estimate the quantity or size of a child's required interventions, thereby reducing the need for multiple calculations at a time of anxiety.

### CROSS-REFERENCE TO RELATED APPLICATIONS

The present Utility patent application claims priority benefit of the U.S. provisional application for patent 60/670,428, filed Apr. 13, 2005 under 35 U.S.C. 119(e). The contents of this related provisional application are incorporated herein by reference.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### FIELD OF THE INVENTION

The present invention relates generally to emergency medical equipment. More particularly, the present invention relates to an apparatus for rapid pediatric resuscitation and/or emergency management of serious pediatric medical conditions in the hospital, emergency room, pediatric clinic, ambulance or aircraft ambulance.

### BACKGROUND OF THE INVENTION

Every year thousands of infants, children and adolescents present to medical facilities in extremis and require resuscitation performed by physicians and medical staff. “Resuscitation” generally refers to interventions undertaken to secure pediatric cardiovascular stability, and includes techniques to secure the patient's airway, improve his/her cardiac function and blood perfusion. Such interventions would usually be required after experiencing conditions including, but not limited to: cardiac compromise, arrhythmias, infection, respiratory compromise, shock, seizure, trauma or other potentially life threatening events. Resuscitation, in these cases, could involve placement of an endotracheal tube to maintain an airway, the administration of blood products and intravenous fluids; medication administration; defibrillation or cardioversion and active re-warming. What is needed is an approach to save lives that increases the accuracy and speed of medical interventions and reduces error, thereby creating improved conditions for resuscitation.

In order to fully appreciate the present invention, it is first helpful to describe the conventional techniques of administering pediatric resuscitation medications. Conventionally, when an ill child presents to medical care, he/she is weighed on a scale at triage. Alternatively, when the child is too ill to stop at the triage desk, an estimate of the child's weight is made on the basis of age, or alternatively, using measurement of the length of the child as a surrogate for weight. Notation of this weight is then made in the chart. In the pre-hospital emergency setting, weights are often estimated by paramedics on the basis of patient age. When a child requires pediatric resuscitative therapy, the noted weight must be used to manually calculate appropriate medication, fluid and electrical therapy dosage and equipment size. The selection of appropriate resuscitative equipment depends on weight determination. For example, the selection of the appropriate-sized endotracheal tube, laryngoscope, and other emergency equipment depends upon the size and weight of the child.

Pediatric resuscitation is more complex than adult resuscitation because medication, blood, cardioversion and intravenous fluid dosages and equipment sizes can vary widely. This is because body mass varies widely between the extremes of the pediatric population. Most adults receive a standard dose of medications for example, because the mass of most adults is in excess the maximum recommended dose of a given medication calculated by weight. In pediatrics, however, medication doses, fluids and equipment sizes are determined on the basis of patient weight, as evidenced by guidelines published by the American Heart Association's Pediatric Advanced Life Support program, and by references such as the Physician's Desk Reference, both which list interventions on the basis of patient weight.

The variation in weight, along with corresponding, variation in dosage has been shown to result in a significant number of errors in pediatric resuscitation. In one recent study, published in the journal Pediatrics, researchers found that approximately 10% of pediatric patients who are seen in a pediatric emergency department were subjected to medication errors and that medication errors were more common among seriously ill patients. These findings were consistent with previous studies identifying dosage calculations as the most frequent reason for medication errors in pediatric patients.

A significant cause of error in emergent settings is the reliance of the physician on estimated weights often made on the basis of the patient's age. Studies have repeatedly shown this to be an ineffective means of determining weight, and the disparity between estimated and actual patient weights can lead to significant under- and over-doses of medications the wrong energy selections while defibrillating or cardioverting patients, and the selection of the inappropriate stabilization equipment such as airway devices.

Another cause of error is the inappropriate recollection of dose per unit weight. At this time, there are several known approaches employed by pediatric and emergency specialists to assist with pediatric resuscitation. Traditional approaches include rote memorization of the required medications and appropriate, memory adjuncts such as reference cards and tables, and the Broselow Pediatric Emergency Tape which lists standard resuscitation medications and their dosages on a tape corresponding to the patient's height, which is used as a surrogate for weight when weight is unavailable.

Next, there is potential for error in resuscitation when staff are asked to convert a requested dose in milligrams to a corresponding volume of medication. This is primarily due to the fact that most resuscitation medications are manufactured in different concentrations and dosage forms, and thereby lack dosage and/or concentration uniformity. For example, drug dosages are customarily provided in milligrams/kilogram: Thus, the first step in medication administration involves hand- or calculator-calculations of dose, on the basis of the child's weight. A second step involves the calculation of the volume of medication to be administered to the child. Resuscitation medications are available in a preselected concentration by the drug manufacturer. Because every resuscitative medication has its own dosage and concentration, the above two-step calculation must be performed for almost every medication administered. The opportunity for error is significant given that these calculations need to be performed quickly, and in a noisy and stress-filled environment. Particularly in the case of a busy emergency department, where staff may only occasionally deal with a pediatric crisis, this invention would improve accuracy and compliance with the recommendations of professional academies. Attempts have been made to develop systems by which standardized concentrations of medications can be administered. In these systems, color-coding is used to identity medications on the basis of concentrations and thus fixed-volumes of medication can be given on the basis of weight without the need to perform sequential dose-concentration calculations. A recent study in the British Medical Journal assessed drug concentrations in syringes administered during a mock pediatric resuscitation found that in 16 percent of 58 syringes analyzed, measured drug concentrations showed a deviation of at least 20 percent from the ordered dose. A large deviation of at least 50 percent from the expected dose was found in 7 percent of cases. This suggests that dose-volume conversions are common and problematic.

There is also potential for error in pediatric resuscitation when physicians and hospital staff fail to adhere to resuscitation guidelines such as those published by the Pediatric Advanced Life Support program and other agencies. These guidelines reflect the work of consensus panels and are regularly updated to reflect best practices. Physicians are not obligated to follow these guidelines, but most choose to do so. Often, physicians intend to follow the guidelines, but are unable to do so because they have forgotten the recommended sequence of medications and resuscitative techniques. This occurs because the management of critically ill children is an infrequent occurrence for many physicians, and infrequent use of resuscitation guidelines may lead to lack of familiarity. This may also reflect the relative complexity of these guidelines.

There is also potential for error in resuscitation when physicians and care-givers become absorbed in the resuscitation and fail to administer medications at regular intervals, as proscribed in current pediatric resuscitation guidelines. For example, epinephrine should be administered every 3-5 minutes in the case of cardiac arrest, yet in many cases this interval is not adequately measured and the medication is given either too frequently, or at intervals too far apart. This could result in serum levels of medication that are too high, or conversely too low.

In view of the foregoing, there is a need in the art for an apparatus, which accurately informs the physician and care team of the appropriate dosages of medication, intravenous fluids and blood, as well as the appropriate equipment selection on the basis of patient weight in the case of critical pediatric illness.

### BRIEF DESCRIPTION OF THE DRAWINGS

The present invention is illustrated by way of example, and not by way of limitations in the figures of the accompanying drawings and in which like reference numerals refer to similar elements and in which:

FIG. 1 illustrates a schematic side view of exemplary weighing, computation and display aspects of the proposed apparatus, in accordance with an embodiment of the present invention;

FIG. 2 illustrates a top section view of an exemplary pediatric treatment apparatus, in accordance with an embodiment of the present invention;

FIG. 3 illustrates a side-view of an exemplary pediatric treatment apparatus, in accordance with an embodiment of the present invention;

FIG. 4 illustrates a front-view of an exemplary pediatric treatment apparatus, in accordance with an embodiment of the present invention;

FIG. 5 illustrates a side-view of an exemplary pediatric treatment apparatus, in accordance with an embodiment of the present invention; and

FIG. 5A illustrates a side view of an exemplary pediatric treatment apparatus, which provides a heated surface for the patient, in accordance with an embodiment of the present invention.

Unless otherwise indicated illustrations in the figures are not necessarily drawn to scale.

### SUMMARY OF THE INVENTION

To achieve the forgoing and other objects and in accordance with the purpose of the invention, a pediatric resuscitation apparatus is described.

One aspect of the present invention provides for the relatively accurate dosing of resuscitation medications and of IV and blood fluids. By integrating weight-determination apparatus, the device provides the practitioner with relatively accurate information concerning the appropriate dosage of a medication and the appropriate volume of resuscitative fluids. Relatively accurate and integrated weight determination also allows for an accurate decision to be made concerning the size of needed resuscitative equipment.

Another aspect of the present invention is to provide a medical apparatus that may simplify decision-making at the time of pediatric resuscitation by reducing the need to estimate the quantity or size of a child's required interventions, and by reducing the need for multiple calculations at a time of anxiety. With the present invention it is hoped to save lives by increasing the accuracy and speed of medical interventions and by reducing error, and by creating improved conditions for resuscitation. When a child presents for emergency treatment, for example, an apparatus embodiment of the present invention would be immediately employed by the physician or other healthcare worker. By placing the child on the apparatus, a relatively accurate weight would be determined. Then, the apparatus would immediately display appropriate dosages of common emergency drugs in milligrams and, given a known concentration of medication used, a volume of medication as well. Another aspect of the present invention is to display appropriate equipment size on the basis of weight, including but not limited to endotracheal tubes, laryngoscope blade size, nasogastric, foley and central venous catheter sizes. In this way, immediate information concerning the child's requirements would be available without the need for reference guides, and mathematical calculations and the chance of calculation error is minimized. Optimum conditions for resuscitation are provided by this device, which may include, for example and not limitation, a firm work surface, warming element and medical cabinetry in various embodiments.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein. Embodiments of the invention are discussed below with reference to the Figures. However, those skilled in the art will readily appreciate that the detailed description given herein with respect to these figures is for explanatory purposes as the invention extends beyond these limited embodiments.

The resuscitation of an ill child is a complex undertaking which requires a great amount of care and which is prone to error and miscalculation. Certain embodiments of the present invention are based on the concept that medications, intravenous fluids, blood products and medical devices used for pediatric resuscitation (pediatric resuscitative therapy) should be as easy to use as possible thus allowing the physician and care team to devote the maximum amount of life-saving attention to the patient while minimizing the chance of error. The present invention provides apparatus embodiments that greatly facilitate rapid and relatively accurate resuscitation treatment of the pediatric patient.

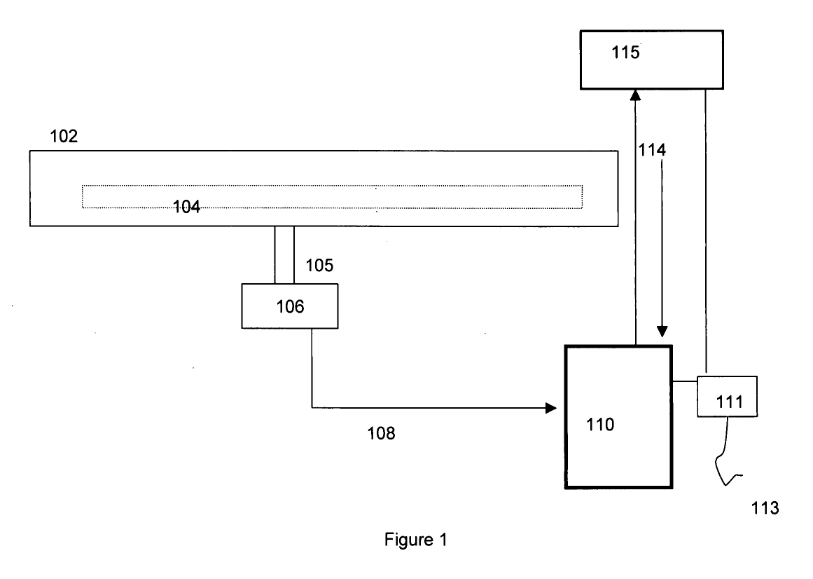

FIG. 1 illustrates a schematic side view of exemplary weighing, computation and display aspects of the proposed apparatus, in accordance with an embodiment of the present invention. The present embodiment comprises a flat patient surface 104 upon which a patient would be placed. Flat patient surface 104 provides a surface upon which resuscitative techniques, such as intravenous placement and airway control, could be performed. This may be constructed of a rigid material such as, but not limited to plastic. Those skilled in the art, in light of the teachings of the present invention, will recognize a multiplicity of alternative and suitable materials for flat patient surface 104, for example, without limitation, metal, hard rubber or sealed wood. Flat patient surface 104 would be surrounded by a supporting frame 102 which may be constructed of a rigid material such as, but not limited to plastic or metal, or as a rigid surface covered in rubber or other impermeable material such as, but not limited to resin, metal, polymer, synthetic or plastic. The mass of the patient would be transferred via a rod 105 or other device, such as, but not limited to a rail, dowel, pipe or other rigid item to a weight transduction apparatus 106 capable of registering the mass of the patient and converting this into an electronic measurement. Weight transduction apparatus 106 could be a strain-gauge transducer or other similar transduction device, such as, but not limited to a load cell or other detection device capable of converting a measured strain or the physical position of rod 105 into an electrical or electronic signal. Weight transduction apparatus 106 would not be limited to one transducer; more than one transducer may be used to electronically determine weight. In the present embodiment, weight readings would be transferred via a wire 108 or electronic medium to a microprocessor 110. Microprocessor 110 would receive input from weight transduction apparatus 106, and from user input entered from input device 114. Input device 114 may be a device such as, but not limited to, a touch-screen monitor, keyboard or similar data entry device. Output from microprocessor 110 is sent to a monitor 115 for display. In some embodiments, the monitor may also function as the data entry device, capable of interpreting provider touch on the screen as an input command. Other data entry devices may be used on other embodiments, including keypads, keyboards and mouse entry devices. In the present embodiment, microprocessor 110 is loaded with current pediatric resuscitative algorithms and would prompt the medical practitioner to administer the next sequential medication or medical intervention according to the algorithm. Microprocessor 110 would also be loaded with the different drug concentrations in use at that particular health care facility in order to perform the calculations needed to determine the correct medication dosage for the patient according to the patient's weight. Microprocessor 110 and other electronic apparatus included in the present invention is powered by attached battery 111 and by connection to electronic mains 113.

In a typical application, the patient, who is a child, is placed on flat patient surface 104. Then weight transduction apparatus 106 ascertains the patient's weight and displays the weight on monitor 115. The medical practitioner then determines the type of treatment needed by the patient, i.e. medication, intravenous fluids, resuscitation procedures, etc. the medical practitioner then uses input device 114 to select the algorithm or medication appropriate for the treatment. Microprocessor 110 then processes this information along with the patient's weight and displays on monitor 115 the appropriate dosage of medication or type and size of equipment to perform the treatment. The apparatus would not recommend a drug dosage if such dosage exceeds the safe or adult dosage. Instead, monitor 115 would alert the practitioner that the dosage is above the maximum dosage and recommend the safe dosage. Monitor 115 will also allow resuscitation prompts and instructions to be displayed according to current accepted medical guidelines. In some embodiments, the prompts could be verbal, or the practitioner could acknowledge administration of the medication using a graphic interface. Monitor 115, in addition to displaying the total volume of medication to be administered, may also remind the practitioner of upcoming medication administration when dosing is recommended at regular intervals. Microprocessor 110 may also provide relatively accurate drip rates when medications are to be given over a certain period of time. Such drip rates refer to volume of medication per minute or hour.

The present invention may be used in numerous situations. For example, without limitation, when a patient is septic and dehydrated, when the patient is in cardiac arrest, or when the patient is in respiratory distress. In the case where the patient is septic and dehydrated, the patient would require antibiotics and intravenous (IV) fluids. The practitioner selects the name of the antibiotics and the name of the IV fluid using input device 114. Monitor 115 informs the practitioner of the volume of IV fluid, and the number of millimeters to be administered per minute as well as the total volume and volume per minute of the antibiotic.

In the case where the patient is in cardiac arrest, the practitioner selects the appropriate algorithm from the device's graphic display, using input device 114. Monitor 115 displays the appropriate airway size and recommends the appropriate volume of medications to be administered according to the cardiac arrest algorithm performed by microprocessor 110. Medical staff acknowledge administration of the medication by signaling via the graphic display. The physician is reminded of the need for an additional medication to be administered by prompting of monitor 115 which displays the subsequent volume to be administered. The patient is kept warm by the apparatus. At the conclusion of the resuscitation, a record of the resuscitation is made available.

In another example, a patient in respiratory distress, is brought to the physician, and is placed on flat patient surface 104. In this case, the device determines the patient's weight and displays this on monitor 115. The practitioner decides to intubate the patient and looks at monitor 115. Monitor 115 displays the appropriately sized endotracheal tube and other airway equipment on the basis of weight. The patient is intubated, on the device, and, in this embodiment, is wheeled to X-ray still on the device. The patient may be connected to oxygen tanks attached to the device as a possible embodiment.

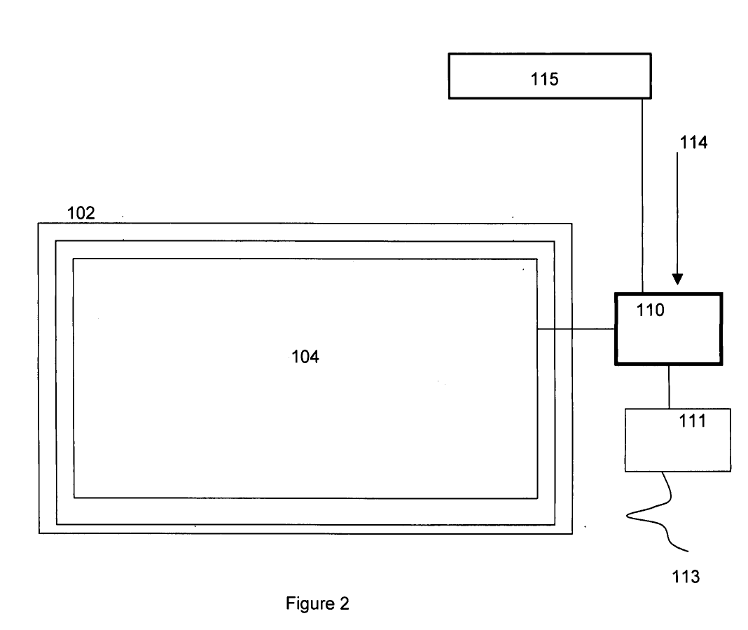

FIG. 2 illustrates a top section view of an exemplary pediatric treatment apparatus, in accordance with an embodiment of the present invention. Flat patient Surface 104 is where a patient would be placed. In the present embodiment, flat patient surface 104 could be constructed of a rigid material such as, but not limited to, plastic or metal, wood, or hard rubber. Flat patient surface 104 is surrounded and under-lied by supporting frame 102 which may be constructed of a rig,id material such as, but not limited to plastic or metal, or of another firm material covered in an impermeable material, such as, but not limited to rubber, plastic or paint. In the present embodiment, the pediatric treatment apparatus also comprises microprocessor 110, battery 111, connection to electrical mains 113 and monitor 115. Input device 114 represents the mechanism by which a user inputs commands to microprocessor 110. In one embodiment input device 114 may be a mouse or keyboard; in another embodiment input device 114 may be the commands from a touch-screen monitor.

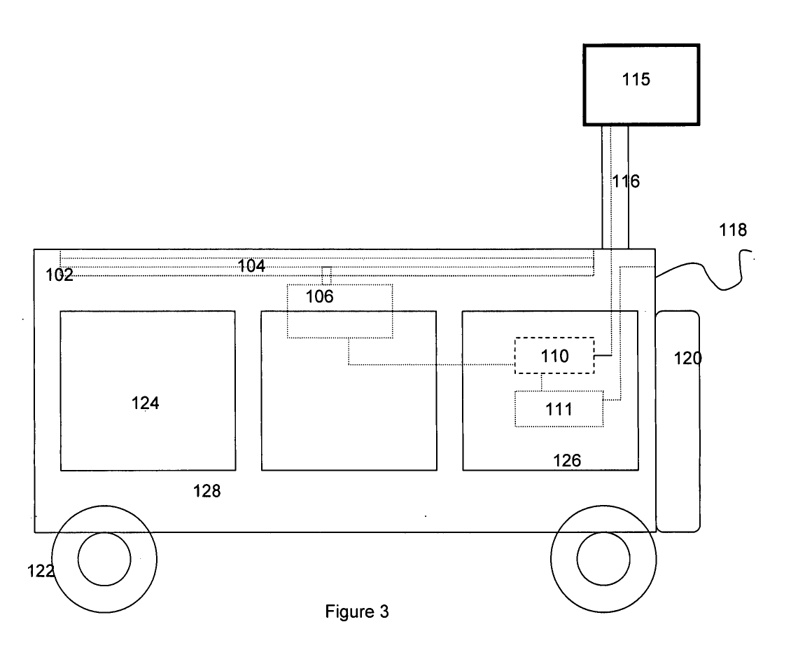

FIG. 3 illustrates a side-view of an exemplary pediatric treatment apparatus, in accordance with an embodiment of the present invention. In the present embodiment, the apparatus comprises a rigid supporting frame 102 constructed of a rigid material, such as, but not limited to, metal or plastic. In the present embodiment, flat patient surface 104 is inlayed into supporting frame 102, and flat patient surface 104 is where the would be placed during treatment. Weight transduction unit 106 senses the weight from the patient and conveys this to microprocessor 110. In the present embodiment supporting frame 102 is surrounded by a layer of cabinetry 128 which may allow for the storage of medical supplies. Cabinetry 128 would be constructed of a rigid material such as, but not limited to metal or plastic. The present embodiment may include securing brackets 120 for portable oxygen cylinders. There is access to the microprocessor 110 via an access panel 126. Access panel 126 would also contain battery 111 and power processing unit, not shown. A wire lead 118 would allow attachment to the electrical mains. In the present embodiment, the apparatus would preferably be mounted on wheels or castors 122 allowing easy portability. Monitor 115 which displays weight, medication, instrument, and treatment information, would preferably be mounted on a telescoping and rotating support 116.

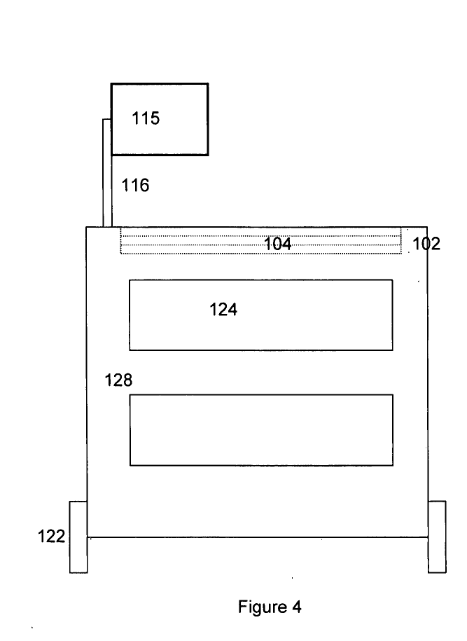

FIG. 4 illustrates a front-view of an exemplary pediatric treatment apparatus, in accordance with an embodiment of the present invention. The present embodiment comprises flat patient surface 104, supporting frame 102, cabinetry 128 and castors or wheels 122. In the present embodiment, monitor 115 is preferably mounted on a telescoping and rotating support 116.

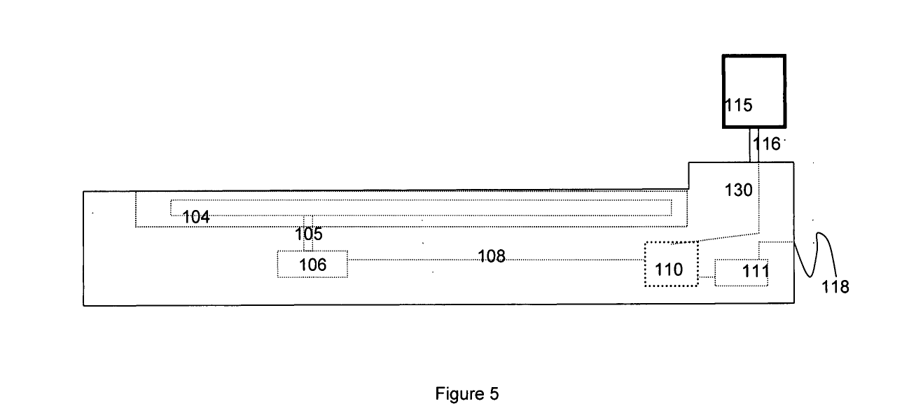

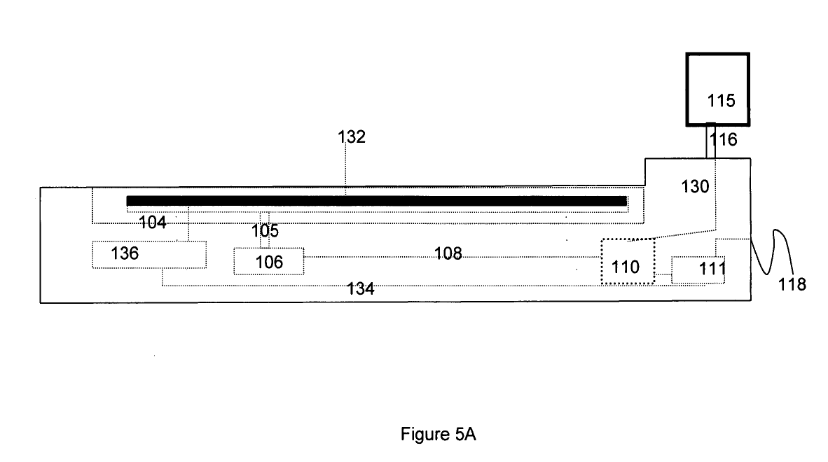

FIG. 5 illustrates a side-view of an exemplary pediatric treatment apparatus, in accordance with an embodiment of the present invention. In the present embodiment, the apparatus may be placed upon a gurney, cot or table, to be used at a comfortable height for the medical practitioner. This height may be at or near the level of the waist of the medical practitioner. The present embodiment comprises a flat patient surface 104 as previously described in FIGS. 1 through 4, a monitor 115 and a supporting mechanism 116 for monitor 115. There is also a microprocessor 110, an electric lead 118 to connect the apparatus to electrical mains in addition to a battery 111 and power transformer and processor, not shown. In the present embodiment, a weight transduction mechanism 106 may be coupled to flat patient surface 104 with the use of a coupling device 105. Examples of means appropriate for coupling device 105 include, but are not limited to, a screw, bolt, dowel, rail or rod. Weight transduction device 106 is attached via wire 108 or other connection to microprocessor 110. The entire apparatus is encased in a rigid frame 130 that supports the unit. Rigid frame 130 may, by way of example, and not limitations be composed of metal, plastic or other rigid material. In the present embodiment, the user commands are input via a touch-screen keypad integral to monitor 115. However, those skilled in the art may, in light of the teachings of the present invention, recognize a multiplicity of other appropriate input devices such as, but not limited to a keyboard or mouse.

FIG, 5A illustrates a side view of an exemplary pediatric treatment apparatus, which provides a heated surface for the patient, in accordance with an embodiment of the present invention. In the present embodiment, a heated element 132 is positioned under or in flat patient surface 104. Heated element 132 may be controlled by a thermostat 136 and heat control equipment, which maintains heat at a pre-determined setting. Heating element 132 and thermostat 136 are connected by a wire 134 to the unit's battery/power control unit 11 and connection to electrical mains 118.

In an alternate embodiment of the present invention, the heated patient surface, as described in FIG. 5A may be integrated into the portable embodiment of the apparatus, as described in FIGS. 1 through 4.

In another embodiment, the microprocessor would maintain a record of all medications and treatments given to the patient suitably referenced (e.g., by time) the patients medical records. The microprocessor could be connected to a standard printer, allowing a printout of the resuscitation record for the patient chart. In another embodiment, the device's microprocessor would connect to the hospital's wireless data system allowing data to be sent to the electronic medical record.

In another preferred embodiment, the device would be comprised of a contained unit, which could be folded via a hinge, to allow for easier storage for use by emergency medical services providers in ambulances or emergency helicopters.

Having fully described at least one embodiment of the present invention, other equivalent or alternative techniques of implementing a pediatric treatment apparatus according to the present invention will be apparent to those skilled in the art. The invention has been described above by way of example, and the specific embodiments disclosed are not intended to limit the invention to the particular forms disclosed. The invention is thus to cover all modifications, equivalents, and alternatives falling within the spirit and scope of the following claims.

What is claimed is:

1. A medical apparatus for the rapid and relatively accurate resuscitation treatment of a patient, the apparatus comprising:
a flat patient surface for placing a patient upon;
at least one weight measurement device joined to the flat patient surface and configured to be alone or in combination operable for measuring and outputting a patient weight value corresponding to the weight of the patient when on the flat patient surface;
an input device for registering user input;
a display unit for displaying output information to a user:
a microprocessor unit configured to receive said patient weight value and at least a portion of the user input, said microprocessor unit being further configured to output information for display on the display unit.

2. The medical apparatus of claim 1, wherein said input device is selected from the input device group consisting of a touch-screen monitor, a keyboard, and a mouse.

3. The medical apparatus of claim 1, further comprising a supporting frame surrounding said flat patient surface.

4. The medical apparatus of claim 3, wherein said supporting frame is constructed of plastic or metal.

5. The medical apparatus of claim 3, wherein said supporting frame is covered, at least in part, with a rubber, a resin, a metal, a polymer, a synthetic, or a plastic.

6. The medical apparatus of claim 1, wherein said display unit comprises said data entry device.

7. The medical apparatus of claim 1, wherein said at least one weight measurement device comprises a strain-gauge transducer or a load cell.

8. The medical apparatus of claim 1, wherein said flat patient surface is constructed of a sufficiently rigid material to properly support the patient.

9. The medical apparatus of claim 8, wherein said rigid material comprises a metal, a hard rubber, or a sealed wood.

10. The medical apparatus of claim 1, further comprising means for transferring the patient's weight to said at least one weight measurement device.

11. The medical apparatus of claim 1, further comprising a computer program product residing on a computer readable medium having a plurality of instructions stored thereon which, when executed by the microprocessor unit, cause that microprocessor unit to:
perform pediatric resuscitative algorithms; and
prompt a medical practitioner to administer a medication or medical intervention according to said algorithm.

12. The medical apparatus of claim 1, further comprising a computer program product residing on a computer readable medium having a plurality of instructions stored thereon which, when executed by the microprocessor unit, cause that microprocessor unit to maintain a record of at least some medications and treatments given to the patient.

13. The medical apparatus of claim 12, wherein the microprocessor is in communication with a printer for generating a printout of a resuscitation record for the patient chart.

14. The medical apparatus of claim 1, further comprising a wireless data system enabling the microprocessor unit to communicate medical information regarding the patient to a medical facility's electronic medical record system.

15. The medical apparatus of claim 1, further comprising a computer program product residing on a computer readable medium having a plurality of instructions stored thereon which, when executed by the microprocessor unit, cause that microprocessor unit to:
perform the calculations needed to determine the correct medication dosage for the patient according to the patient's weight, the calculations being based, at least in part, on drug concentrations in use at that particular health care facility.

16. The medical apparatus of claim 1, further comprising a gurney, cot or table that supports said medical apparatus at a convenient height for a medical practitioner.

17. The medical apparatus of claim 1, further comprising at least one heating element is disposed under or in said flat patient surface.

18. In another preferred embodiment, the device would consist of a contained unit, which could be folded via a hinge, to allow for easier storage for use by emergency medical services providers in ambulances or emergency helicopters.
